(19) United States
(12) Patent Application Publication
(10) Pub. No.: US 2023/0276798 A1
(43) Pub. Date: Sep. 7, 2023

(54) METHOD AND COMPOSITION FOR PATHOGEN INHIBITION UTILIZING ENGINEERED CRYSTALLINE STRUCTURES

(71) Applicant: Strategia Project Management, LLC, an Illinois Limited Liability Company, Oak Brook, IL (US)

(72) Inventor: Erwin LO, Spring, TX (US)

(73) Assignee: Strategia Project Management, LLC, an Illinois Limited Liability Company, Oak Brook, IL (US)

(21) Appl. No.: 18/024,451

(22) PCT Filed: Sep. 2, 2021
(86) PCT No.: PCT/US2021/048853

§ 371 Date: Mar. 2, 2023

Related U.S. Application Data
(60) Provisional application No. 63/073,881, filed on Sep. 2, 2020.

(51) Int. Cl. A01N 33/12; A01N 25/02; A01P 1/00

(52) U.S. Cl. CPC A01N 33/12; A01N 25/02; A01P 1/00

(57) ABSTRACT

An engineered crystalline structure created from a solution containing silica dioxide that is applied on a surface and dries forming a barrier coating on the treated surface. The formed of the barrier coating has proven to be an effective antimicrobial barrier solely with inert ingredients. Moreover, the established barrier has been shown to eradicate SARS-CoV-2 within two hours without chemical intervention. When applied to a surface and allowed to dry, a 4-6 nm crystalline-like layer of silica dioxide bonds covalently to the surface. The crystalline-like structures making up the “new” surface are invisible to the naked eye but can be observed via a high-powered electronic microscope. Once silica dioxide is applied to a surface, the crystalline-like structures contain spikes that penetrate the outer membranes and protein coats of microorganisms, rendering them dead in the case of bacteria and fungi or inactive in the case of viruses.

### CROSS-REFERENCE TO RELATED APPLICATION

This application claims benefit of U.S. Provisional Application No. 63/073,881, filed Sep. 2, 2020, which is incorporated herein by reference in its entirety for all purposes.

### FIELD

The present disclosure generally relates to cleaning and disinfectant coatings for surfaces, and in particular surface coatings for the reduction of viruses.

### BACKGROUND

A novel coronavirus was first reported in Wuhan City, Hubei Province, China and later named severe acute respiratory syndrome coronavirus 2 (SARS-CoV-2). Coronaviruses are a family of RNA viruses that have been previously identified as having six subtypes; SARS-CoV-2 is now classified as a seventh subtype. SARS-CoV-2 spreads more efficiently than SARS-CoV (which became more generally known in 2003 simply as SARS) and MERS-CoV (which became more generally known in 2015 simply as “the MERS virus”) and causes the disease now generally referred to as “coronavirus 2019” or more generally, COVID-19. SARS-CoV-2 is principally spread through respiratory secretions or droplets expelled by infected individuals onto surfaces and objects thereby creating fomites (contaminated surfaces).

Viable SARS-CoV-2 virus and/or RNA detectable by RT-PCR can be found on those surfaces for periods ranging from hours to days depending on the ambient environment conditions (including such aspects as temperature and humidity levels) and the type of surface so contaminated. Sadly, particular high incidents of SARS-CoV-2 contamination has been detected in facilities where COVID-19 patients reside or are being treated for the disease, including convalescence homes for the elderly, emergency rooms and hospitals. Accordingly, transmission of SARS-CoV-2 can occur indirectly by persons touching such contaminated surfaces and then contacting their mouth, nose, or eyes where the virus finds ease of entry, infecting that person.

Consequently, there is a need for non-irritating, antimicrobial (efficacious against bacteria and viruses) substances that have residual effectiveness against harmful pathogens for relatively long periods of time. Currently, the majority of antimicrobial or biocidal agents are only effective while they are wet and traditionally have necessarily contained harsh, irritating and potentially harmful chemicals as their active ingredient(s). Therefore, products that do not require such harmful chemicals for effective pathogen control, over long periods of time, provide great user benefits.

### BRIEF DESCRIPTION OF THE DRAWINGS

FIG. 1 is a 3-dimensional graphic by an electron microscope at 100× magnification of a glass coverslip (slide) with no treatment applied;

FIG. 2, in the top portion, is a 3-dimensional graphic by an electron microscope at 100× magnification of a glass coverslip with one treatment of colloidal silica dioxide coating configured according to the present disclosure and applied to an edge of the coverslip; in the bottom portion, a line scan height analysis is illustrated;

FIG. 3 is a 3-dimensional graphic by an electron microscope of a glass coverslip with one treatment of colloidal silica dioxide coating configured according to the present disclosure and applied to an edge of the coverslip and scratched with a utility knife;

FIG. 4 is a 3-dimensional graphic by an electron microscope of a glass coverslip with one treatment of colloidal silica dioxide coating configured according to the present disclosure and applied to an edge of the coverslip and scratched with a utility knife; and

FIG. 5 is a bar graph showing the engineered silica solution coating (also referred to as Microsure in FIG. 5) without any added chemicals successfully eradicated 100% the SARS-CoV-2 virus (left column of each pair) in under 60 minutes compared to the water control (right column of each pair).

### DETAILED DESCRIPTION

The presently disclosed technology for producing antimicrobial products was tested on SARS-CoV-2, as well as other surrogate viruses. It proved to have statistically significant efficacy against the virus, as well as long-term residual antimicrobial effects.

This technology establishes an elemental colloidal silica dioxide coating that provides a barrier against pathogenic microbes. While the use of silica in combination with certain metals such as silver, copper and gold has been previously described as having certain antimicrobial effects, the present technology of an engineered nanomolecular colloidal silica that itself has antimicrobial effect and potentiates the effect of other active antimicrobial ingredient(s) when included in minimal amounts.

The engineered crystalline structures created in accordance with the presently disclosed technology are visually apparent under high powered electron microscopy after the silica dioxide-containing solution is applied on a surface and dries forming a barrier coating on the now-treated surface. Uniquely, the formed barrier coating has proven to be an effective antimicrobial barrier solely with inert ingredients. Moreover, the established barrier has been shown to meet a baseline of log 3 viricidal reduction within two hours. When applied to any solid surface and allowed to dry, a 4-6 nm crystalline-like layer of silica dioxide bonds covalently (permanently) to the substrate. The crystalline structures making up the “new” surface” cannot be seen with a naked eye but can be observed via a high-powered electronic microscope. Once silica dioxide is applied to a surface, the crystalline-like structures that make up the resulting “modified” surface contain microscopic spikes that physically work to penetrate the outer membranes and protein coats of microorganisms that come into contact with the surface, rendering them dead in the case of bacteria and fungi or inactive in the case of viruses.

It is known that chemical kill mechanisms are only short-term effective. The short-term limitation stems from the inactivation of the chemical kill once the chemical agent has dried, which may be mere seconds after application. On the other hand, a mechanical kill such as that of the silica dioxide matrix lasts for as long as the mechanical agent remains on the surface. As a result, the product is effective both at the time of application and well after. The presently disclosed composition embodies a silica dioxide matrix that, once applied, forms microcrystalline structures that form a covalent bond with the targeted surface. This mechanical barrier protects the surface from adherence of harmful microbes by way of the crystalline structures' inclusion of the microscopic spikes that penetrate the outer membranes of the microbes, rendering them unable to attach to the surface. This barrier can remain on the surface for an extended period.

The nanotechnology associated with the presently disclosed silica dioxide solution is able to attach to surfaces via covalent bonds, thereby creating a physical “pesticide device” barrier that lasts on surfaces for an extended period of time. Moreover, extended antimicrobial effects have been shown to exist after 24 hours.

The unique soluble antimicrobial solutions of the present disclosure are suitable to protect multiple diverse surfaces including both inanimate surfaces and human skin. The present technology has particular utility in the public transportation industries that are concerned about the spread of microbes to passengers, including airlines and cruise lines. To facilitate application, aerosol delivery systems are utilized for such large area coverage needs.

In addition, the surface protestant's ability to persist over time has been specifically demonstrated in multiple wash tests that show that the barrier persists on textiles through numerous washings. For example, a textile study examined the efficacy of the protectant on multiple textiles, including socks, towels, and medical scrubs, while undergoing numerous commercial wash cycles. Biocidal activity was present on the treated fabric after application. The solution continued to be effective after several cycles, with some materials showing efficacy at up to 75 commercial washes.

An observational, non-randomized, open labeled, mono-centric, controlled experiment was conducted in which the mechanical kill efficacy against SARS-CoV-2 was evaluated using the presently described nanomolecular colloidal silica dioxide surface protectant and hand sanitizer. The experiment was divided into two testing paradigms: (1) antiviral effect on a so-coated glass coverslip tested after the addition of viral pathogen cells and (2) antiviral effect of coated glass coverslips tested prior to addition of viral pathogen cells. Tested samples included: (1) hand sanitizer comprising (including, but not necessarily limited to) benzalkonium chloride (BZK) and the nanomolecular colloidal silica dioxide; (2) surface protectant comprising quaternary ammonium (QAC) and the nanomolecular colloidal silica dioxide; (3) the nanomolecular colloidal silica dioxide with no additional active ingredients and (4) a control of deionized water.

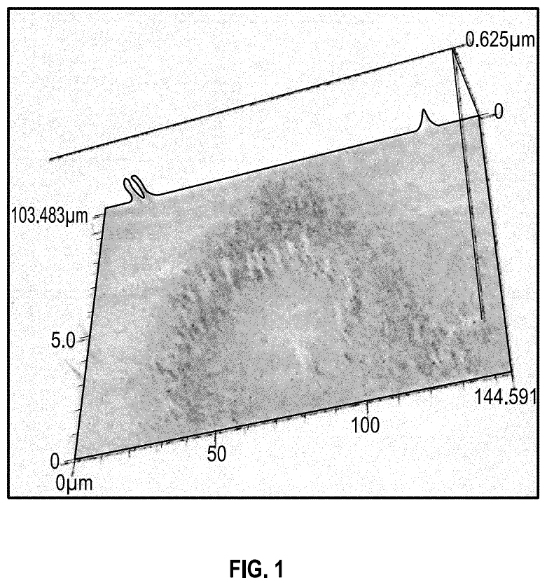

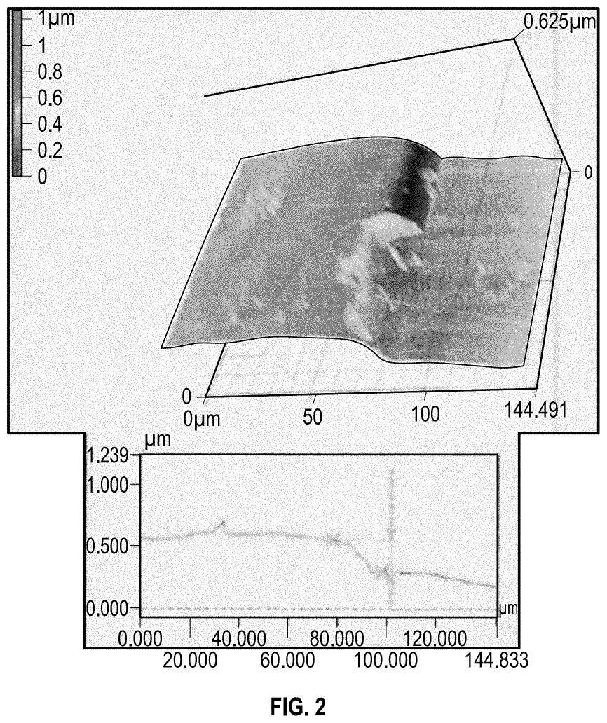

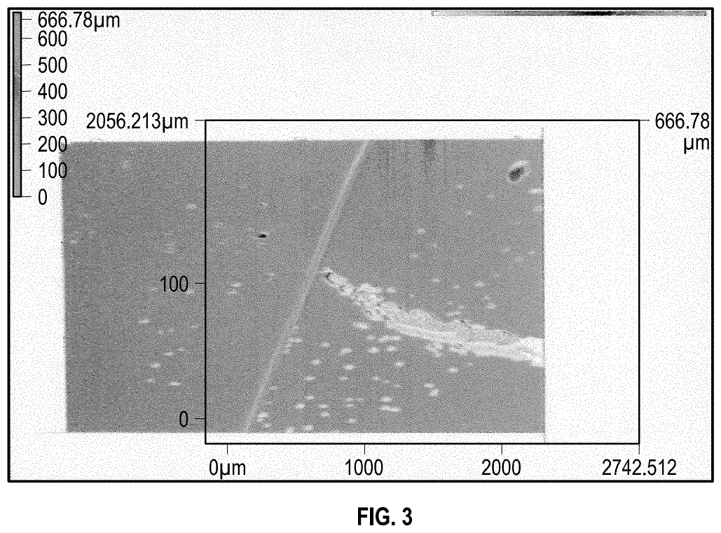

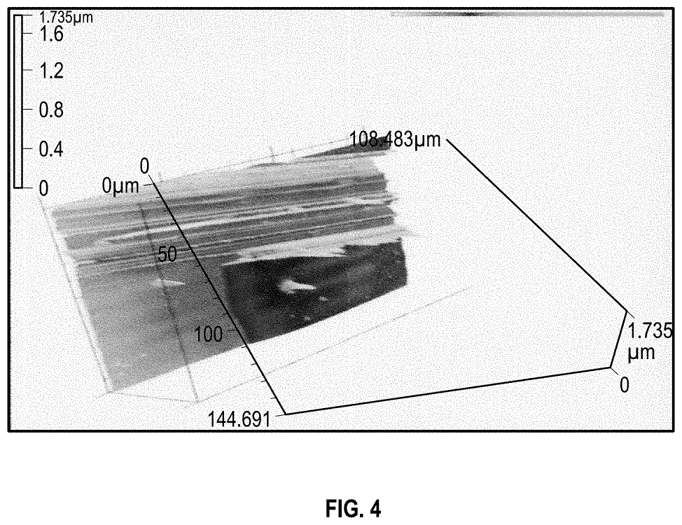

FIG. 1 and FIG. 2 depict a glass slide before and after application of a nanomolecular colloidal silica dioxide coating containing silica dioxide. These images clearly depict the physical, three-dimensional coating created following application of the silica dioxide solution. In addition, laboratory testing has demonstrated that use of a razor, applied with force, is required to remove the crystalline-like barrier from a treated surface. The images of FIG. 3 and FIG. 4 depict a slide treated with a silica dioxide solution according to the disclosed technology and subsequently scratched with a utility knife which shows that even when abraded off with robust force using a blade, there is still residual silica present forming a pathogen barrier.

The demonstrated ability of silica dioxide to form a covalent bond with surfaces has multiple demonstrated and potential benefits in, among others, the healthcare field. It has been shown that the use of silica dioxide in accordance with this disclosure can have positive effects related to cellular function and therefore skin-related function, provides benefits in bone tissue engineering and can increase resistance to moisture and corrosion on surfaces. In addition, a clinical study conducted on the use of the presently disclosed colloidal silica dioxide depicts benefits realized in the treatment of infected wounds. The same mechanism of action demonstrated in these studies also supports the silica dioxide's ability to act mechanically as a pesticide device that physically prevents further entry of unwanted microbes. This makes the solution effective as a surface protectant (internal and external) over an extended period.

In the experiment, under a BSL2 hood, an amount of each of the four test substances was poured into respective small beakers sufficient to permit complete dipping of glass (18 mm×18 mm) coverslips. Using sterile forceps, each coverslip was dipped for 20 seconds into the respective solution and allowed to air dry under sterile conditions. The so-coated coverslips were prepared on the same day that the balance of the experiment was conducted. Evaluation for possible surface modifying properties of the test substances was performed by adding culture media to the coverslip. A dual testing paradigm was developed for the experiment.

TestingParadigm 1—TEST SUBSTANCE AFTER VIRAL PLACEMENT: using plates 0.5×10⁶ L2 cells in 6-well plates. Prepare 3 replicates pertreatment. Allow the cells to attach and grow for 24 hours. Place the cells on ice and wash the cells with cold DME 2 (2% serum). Remove wash and add 4×10² plaque forming units MHV-A59-GFP in 400 ul and allow the virus to attach for 1 hour (this should infect approximately 200 cells on a coverslip in the control group). Remove the virus and wash the cells twice with DME 2. Add 2 ml of cold DME2. Place the coated coverslips inside the wells and keep on ice for 1 minute, 5 minutes, 10 minutes, 60 minutes and 3 hours then transfer to a 37 incubator. After 8 hours of incubation, count the fluorescent cells. Check for GFP fluorescence.

Testing Paradigm 2—PRIOR VIRAL PLACEMENT: Place coated coverslips in 6-well plates. Add 107 PFU of MHV-A59 in 400 ul of DME-2 and keep at 4 degrees Celsius under humidified, sterile conditions for 1 minute, 5 minutes, 10 minutes, 1 hour, 3 hours, 24 hours, 7 days, 14 days and 30 days. Remove the samples and place in sterile Eppendorf tube and freeze at −80 degrees Fahrenheit. Titer virus by plaque assay.

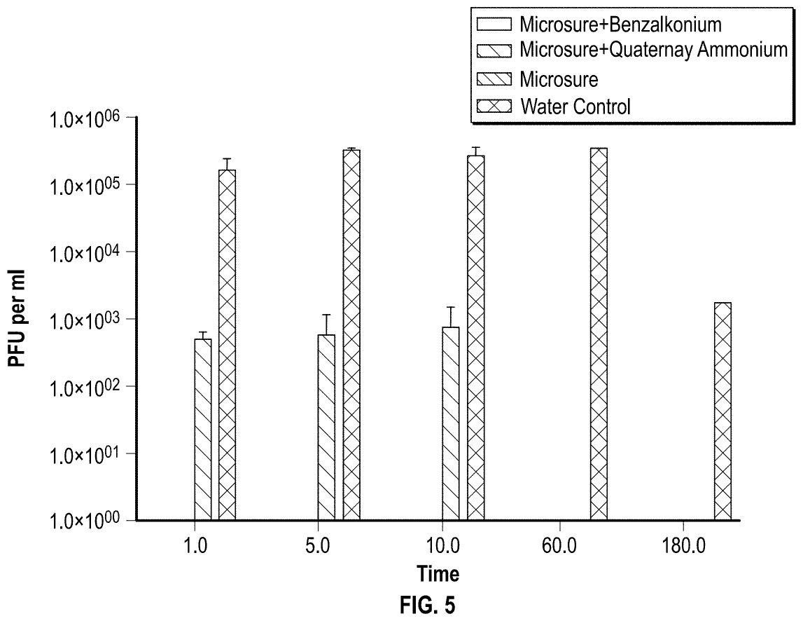

Referring to FIG. 5, where time is shown in elapsed minutes, each of the test solutions can be associated to their matching color or cross-hatching within the legend depicted at the top of the graph. The presently disclosed engineered silica with the (1) benzalkonium chloride and with the (2) quaternary ammonium each successfully eliminated the virus 100% within the first one minute of treatment and as such do not appear in the graph of FIG. 5. The engineered silica solution (also referred to as Microsure in FIG. 5) without any added chemicals successfully 100% eradicated the SARS-CoV-2 virus in under 60 minutes of treatment, demonstrating that all three of the tested solutions exceeded the acceptable coronavirus EPA performance benchmark minimum to be considered efficacious.

These results demonstrate that the chemical components (benzalkonium chloride and quaternary ammonium) of the solutions are almost immediately effective when combined with the present engineered silica solution (Microsure). The engineered silica solution alone, without any other active ingredients, after sufficient time to dry on the surface, created the newly modified barrier of protection and began defending. All-in-all, each of the three tested engineered silica solution effectively eradicated 100% of the applied SARS-CoV-2 virus and continued to do so for the balance of the 30 day test period.

As shown in this study, the two engineered silica solutions with added active ingredient were able to successfully eradicate 100% of the tested coronavirus within one minute and the engineered silica solution alone was able to successfully eradicate 100% of the tested coronavirus in under one hour. Not only were the solutions effective almost immediately but each of the tested solutions continued to protect and remain biocidal throughout the 30-day test period, further proving that the nanomolecular silicon dioxide structures that are formed remain intact and continue to protect long after it has dried on the surface.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

What is claimed is:

1. A composition comprising microsure, microsure having nanomolecular colloidal silica.

2. The composition of claim 1, further comprising Benzalkonium chloride (BZK).

3. The composition of claim 1, further comprising a quaternary ammonium (Quat Ammonia).

4. A process comprising:
applying microsure to a surface to form a coating, microsure having nanomolecular colloidal silica.

5. The process of claim 4 further comprising applying Benzalkonium chloride (BZK) to the surface to form the coating.

6. The process of claim 4 further comprising applying a quaternary ammonium (Quat Ammonia) to the surface to form the coating.

7. The process of any one of the proceeding claims 4-6, further comprising contacting the microsure surface with SARS-Cov-2.
